(19) United States
(12) Patent Application Publication
(10) Pub. No.: US 2022/0125379 A1
(43) Pub. Date: Apr. 28, 2022

(54) METHODS FOR IDENTIFYING RESPONDERS TO DOPAMINERGIC NEURONAL ENHANCING THERAPIES FOR THE TREATMENT OF ELEVATED HEART RATE AND METABOLIC OR CARDIOVASCULAR CONDITIONS

(71) Applicant: VeroScience LLC, Tiverton, RI (US)

(72) Inventor: Anthony H. Cincotta, Tiverton, RI (US)

(21) Appl. No.: 17/569,863

(22) Filed: Jan. 6, 2022

Related U.S. Application Data
(63) Continuation of application No. 15/607,134, filed on May 26, 2017, now abandoned.
(60) Provisional application No. 62/342,076, filed on May 26, 2016.

(51) Int. Cl. A61B 5/00; A61B 5/024; A61B 8/08

(52) U.S. Cl. CPC A61B 5/4839; A61B 5/024; A61B 5/4866; A61B 5/4857; A61B 8/0883

(57) ABSTRACT

Methods for identifying responders to agents that increase brain dopaminergic neuronal activity for the treatment of metabolic and cardiovascular disease and for treating elevated heart rate and metabolic disease or dysglycemia in subjects with type 2 diabetes.

### CROSS-REFERENCE TO RELATED APPLICATIONS

This application claims the benefit of U.S. Provisional Application No. 62/342,076, filed on May 26, 2016. The prior application is incorporated herein by reference in its entirety.

### TECHNICAL FIELD

This disclosure relates to methods for the treatment of elevated heart rate and metabolic disease or dysglycemia in subjects with type 2 diabetes and methods for identifying responders to agents that increase brain dopaminergic neuronal activity for the treatment of metabolic and cardiovascular disease.

### BACKGROUND OF THE INVENTION

The global health crisis of obesity, diabetes, and related metabolic disorders has been well-established since before the turn of this century. The prevalence of type 2 diabetes, obesity, dysglycemia, and other metabolic syndromes is reaching pandemic proportions worldwide, and their prevalence is expected to continue to rise over the next two decades, further exacerbating the health crisis surrounding these diseases. Estimates of people diagnosed with diabetes will likely exceed 350 million globally by 2030 (see, e.g., Wild S, Diabetes Care, 2004, 27: 1047). Diabetes and its associated co-morbidity exact a high toll on both subjects and the healthcare system. In the United States, diabetes represents 11% of total U.S. healthcare expenditures, with cardiovascular disease accounting for approximately 20% of the annual direct medical costs associated with diabetes (see, e.g., http://www.diabetes.org). Despite efforts to reduce cardiovascular risk factors in subjects with diabetes, 65% of subjects with diabetes will die from heart disease and stroke, and the fact remains that type 2 diabetes increases the risk for cardiovascular disease two-fold for men and three-fold for women relative to gender-matched individuals without type 2 diabetes (see, e.g., Conroy, Eur Heart J., 2003, 24: 987). A safe and effective treatment for cardiovascular conditions in subjects with type 2 diabetes would impart significant benefits to humanity.

### SUMMARY OF THE INVENTION

The invention relates to a method of identifying a subject that will be a responder to treatment with agents that increase brain dopaminergic neuronal activity. The method includes the steps of (a) obtaining an echocardiogram of the subject or assessing the subject's pulse rate; (b) determining the subject's resting heart rate using the echocardiogram or the pulse rate; (c) determining that the subject will benefit from treatment with agents that increase brain dopaminergic neuronal activity if the subject's resting heart rate is greater than about 70 beats per minute; and (d) treating the subject with agents that increase brain dopaminergic neuronal activity.

In another embodiment, the invention relates to a method of identifying predicting whether a subject that will be a responder to benefit from treatment with agents that increase brain dopaminergic neuronal activity. The method includes the steps of (a) obtaining an initial echocardiogram of the subject or assessing the subject's initial pulse rate; (b) determining the subject's initial resting heart rate using the initial echocardiogram or the initial pulse rate; (c) administering one or more agents that increase brain dopaminergic neuronal activity to the subject if the subject's initial resting heart rate is greater than about 70 beats per minute; (d) obtaining a new echocardiogram of the subject or assessing the subject's new pulse rate; (e) determining the subject's new resting heart rate using the new echocardiogram or the new pulse rate; and (f) determining that the subject will benefit from treatment with agents that increase brain dopaminergic neuronal activity if the subject's new resting heart rate is lower than the subject's initial resting heart rate.

In another embodiment, the invention relates to a method of treating a subject having one or more metabolic conditions. The method includes the steps of (a) determining whether the subject will benefit from treatment with agents that increase brain dopaminergic neuronal activity according to the methods described in the paragraphs above; and (b) administering to the subject an effective amount of one or more agents that increase brain dopaminergic neuronal activity if the subject will benefit from treatment with agents that increase brain dopaminergic neuronal activity, wherein the one or more metabolic conditions are selected from type 2 diabetes, prediabetes, metabolic syndrome, obesity, hypertension, glucose intolerance, hyperglycemia, insulin resistance, elevated plasma lipid levels, fatty liver disease and cardiovascular disease.

### BRIEF DESCRIPTION OF THE FIGURES

FIG. 1 is a graph depicting results of the study described in Example 3.

### DETAILED DESCRIPTION OF THE INVENTION

The present disclosure provides methods for the treatment of elevated heart rate, metabolic disease, and/or dysglycemia in subjects with type 2 diabetes, and methods for identifying responders to agents that increase brain dopaminergic neuronal activity for the treatment of metabolic and cardiovascular disease.

More specifically, the document provides a method of predicting whether a subject will benefit from treatment with agents that increase brain dopaminergic neuronal activity, the method including obtaining an echocardiogram/electrocardiogram (ECG or EKG) of the subject or assessing the subject's pulse rate; determining the subject's resting heart rate using the echocardiogram/electrocardiogram or the pulse rate; and determining that the subject will benefit from treatment with agents that increase brain dopaminergic neuronal activity if the subject's resting heart rate (“RHR”) is greater than about 70 beats per minute (e.g., greater than about 80 beats per minute).

Moreover, the document provides a method of predicting whether a subject will benefit from treatment with agents that increase brain dopaminergic neuronal activity, the method including obtaining an initial echocardiogram/electrocardiogram of the subject or assessing the subject's initial pulse rate; determining the subject's initial resting heart rate using the initial echocardiogram/electrocardiogram or the initial pulse rate; administering one or more agents that increase brain dopaminergic neuronal activity to the subject if the subject's initial resting heart rate is greater than about 70 beats per minute (e.g., greater than about 80 beats per minute); obtaining a new echocardiogram/electrocardiogram of the subject or assessing the subject's new pulse rate; determining the subject's new resting heart rate using the new echocardiogram/electrocardiogram or the new pulse rate; and determining that the subject will benefit from treatment with agents that increase brain dopaminergic neuronal activity if the subject's new resting heart rate is lower than the subject's initial resting heart rate.

In any of the above-described methods, the subject can have, e.g., type 2 diabetes, prediabetes, metabolic syndrome, obesity, hypertension, glucose intolerance, hyperglycemia, insulin resistance, elevated plasma lipid levels, and/or cardiovascular disease.

Also provided by the document is a method of treating a subject having one or more metabolic conditions, the method including determining whether the subject will benefit from treatment with agents that increase brain dopaminergic neuronal activity according to any of the above-described methods; and administering to the subject an effective amount of one or more agents that increase brain dopaminergic neuronal activity if the subject will benefit from treatment with agents that increase brain dopaminergic neuronal activity. The one or more metabolic conditions can include, e.g., type 2 diabetes, prediabetes, metabolic syndrome, obesity, hypertension, glucose intolerance, hyperglycemia, insulin resistance, elevated plasma lipid levels and/or cardiovascular disease.

The document additionally provides a method of simultaneously treating a metabolic condition and elevated resting heart rate, the method including administering to a subject one or more agents that increase brain dopaminergic neuronal activity so as to effectuate a peak in brain dopaminergic neuronal activity within 4 hours of the onset of the subject's daily waking cycle. The metabolic condition can include, e.g., type 2 diabetes, prediabetes, metabolic syndrome, obesity, hypertension, glucose intolerance, hyperglycemia, insulin resistance, elevated plasma lipid levels and/or cardiovascular disease.

In any of the above-described methods, the one or more agents that increase brain dopaminergic neuronal activity can include, e.g., one or more postsynaptic dopamine receptor agonists (e.g., one or more dopamine D₁ and/or dopamine D₂ receptor agonists).

In any of the above-described methods, the one or more agents that increase brain dopaminergic neuronal activity can include, e.g., bromocriptine.

In any of the above-described methods, the one or more agents that increase brain dopaminergic neuronal activity can be administered to the subject so as to effectuate a peak in brain dopaminergic neuronal activity, e.g., within about 4 hours of the onset of the subject's daily waking cycle.

In any of the above-described methods, the subject can be a diurnal animal (e.g., a human) and the one or more agents that increase brain dopaminergic neuronal activity can be administered to the subject so as to effectuate a peak in brain dopaminergic neuronal activity, e.g., between the time interval of about 0400 and about 1200 hours of the day. The one or more agents that increase brain dopaminergic neuronal activity can be administered to the subject, e.g., between the time interval of about 0400 and about 1200 hours of the day.

As used herein, the terms “about” and “approximately” are defined as being within plus or minus 10% of a given value or state, preferably within plus or minus 5% of said value or state.

The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome (e.g., reduction in occurrence of CVD events).

The term “responder” as used herein refers to an individual that obtains or is expected to obtain a better than population average response to a therapy that increases central dopaminergic neuronal activity.

As defined herein, “neuronal activity” refers to either an increase or decrease in the action potential of a neuron. More specifically, as defined herein, “neuronal activity” refers to either an increase or decrease in the synaptic neurochemical signal transmission of a neuron to another thereby affecting action potential. More narrowly yet, as defined herein, “neuronal activity” refers to the biochemical communication to a (secondary (e.g., post-synaptic)) neuron from either the neurochemical signal transmission of another (primary (e.g., pre-synaptic)) neuron (e.g., as via an endogenous neurotransmitter) or from any neuromodulatory compound (e.g., an exogenous neurotransmitter receptor modulator such as a pharmaceutical agent) thereby affecting action potential or neurotransmitter release of the secondary neuron. As such, an increase in dopaminergic neuronal activity would be characterized by a) an increase in release of dopamine molecules from a dopamine producing (primary) neuron, an increase in dopamine molecules within the synapse by any mechanism, and/or increase in dopamine-mimetic compound(s) from any source (e.g., pharmaceutical) resulting in increased binding to dopaminergic receptor sites of other (secondary) neuron(s) that affect said other neuron(s)′ action potential or neurotransmitter release in a manner consistent with increased dopamine ligand-dopamine receptor binding signal transduction (e.g., post-synaptic dopamine receptor agonist) and/or b) an increase in sensitivity or responsiveness of said “other (secondary)” neuron(s) to such dopamine or dopamine-mimetic compound(s)′ ability to affect action potential or neurotransmitter release in said “other (secondary)” neuron (e.g., as an increase in dopamine receptor number or affinity or responsiveness). Contrariwise, dopamine-mimetic binding to dopamine-producing neurons (i.e., presynaptic dopamine neurons) and/or increased sensitivity or responsiveness of dopamine producing neurons to respond to neurotransmitters or neuromodulators that thereby reduces dopamine release would be considered an activity leading to a decrease in dopaminergic neuronal activity (and, when considered in and of itself, is an undesirable aspect of dopaminergic activity respecting this invention). For the sake of clarity, post-synaptic dopamine receptor agonists include dopamine D1, D2, D3, D4, and D5 receptor agonists

Unless otherwise defined, all technical and scientific terms used herein have the same meaning as commonly understood by one of ordinary skill in the art to which this invention belongs. Methods and materials are described herein for use in the present invention. Other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

Metabolic disorders, including type 2 diabetes mellitus, are exceedingly complex pathologies that involve aberrations in multiple organ systems and cellular level biochemical pathways. While various therapies have been developed that target different specific aspects of these pathophysiological complexities of metabolic disease, what is needed is a method of identifying those subjects with a specific pathophysiology/biochemistry that represents a strong biomarker for identifying individuals that will exhibit enhanced responsiveness to a given therapy for a particular metabolic disorder and thus provide optimal health care to the subject. Circadian timed increase in central dopaminergic neuronal activity, such as with a postsynaptic dopamine receptor agonist, represents a treatment strategy for a variety of metabolic diseases. However, methods of identifying subjects most likely to respond to such a treatment strategy would enhance the clinical utility and therapeutic index (effective to toxic dose ratio) of the therapy and is the basis for subject specific medicine, a most desirable approach to medical treatment of disease. This invention provides for such a method.

Quick-release bromocriptine (BQR) (Cycloset®) is a sympatholytic dopamine D₂ receptor agonist, insulin sensitizer approved for the treatment of type 2 diabetes. It was unexpectedly discovered that administering an agent that increases the brain dopaminergic neuronal activity (e.g., bromocriptine (e.g., BQR)) at the appropriate time of day to type 2 diabetes subjects in poor glycemic control (e.g., subjects with a baseline glycated hemoglobin (HbA1c) level ≥about 7.5%) and with a RHR≥about 70 beats per minute (an “elevated RHR”) significantly reduces the subjects' RHR and HbA1c level. For example, a subject's RHR can be reduced by, e.g., 1, 2, 3, 4, 5, 6, 7, 8, 9, 10, 11, 12, 13, 14, 15, 16, 17, 18, 19 or to 20 or more beats per minute. A subject's elevated RHR level is generally reduced, e.g., within about 14 days of administering BQR. Administering BQR to type 2 diabetes subjects with an even higher RHR (e.g., a RHR≥about 80 beats per minute) reduces the subjects' RHR and HbA1c level by an even greater amount. It was also surprisingly found that elevated RHR identifies those subjects in whom such dopamine activity modulating therapy will produce a much better than average improvement in their metabolic disease. A subject RHR can be determined, e.g., using an electrocardiogram/echocardiogram (EKG or ECG) of the subject or by assessing the subject's pulse rate.

Without wishing to be bound by theory, it is believed that these findings support a central (brain) biological clock mechanism for BQR-induced reduction of both RHR and HbA1c in subjects with elevated RHR.

Alternately or in addition to type 2 diabetes, subjects can have, e.g., one or more of prediabetes, metabolic syndrome, obesity, hypertension, glucose intolerance, hyperglycemia, insulin resistance, elevated plasma lipid levels and cardiovascular disease.

One or more other agents that increase the brain dopaminergic neuronal activity can be administered to a subject, in addition to or in lieu of bromocriptine. Examples of such agents are known in the art and include, e.g., postsynaptic dopamine receptor agonists (e.g., dopamine D₁ agonists and dopamine D₂ agonists), presynaptic dopamine transporter inhibitors, presynaptic dopamine autoreceptor antagonists (e.g., AJ76), presynaptic dopamine release enhancers and dopamine synthesis stimulators (e.g., brain derived neurotrophic factor (BDNF) and L-DOPA), dopamine catabolism/degradation inhibitors (e.g., monoamine oxidase inhibitor B (e.g., deprenyl) and dopamine monooxygenase inhibitor), and dopamine reuptake inhibitors (e.g., GBR-12909, GBR-12935, and GBR-12783). See, e.g., U.S. Pat. No. 9,655,865.

For example, a dopamine D₁ agonist can be any one or more of those compounds known to those skilled in the art that are capable of activating or potentiating D₁ dopamine receptors. Dopamine D₁ agonists suitable for use in the present invention include, e.g., benzazapine, SKF 38393 (1-phenyl-2,3,4,5-tetrahydro-1H-3-benzazepine-7,8-diol), dihydrexidine, SKF 75670 (7,8-dihydroxy-3-methyl-1-phenyl-2,3,4,5-tetrahydro-1H-3-benzazepine hydrobromide), SKF 82957 (C₁₇H₁₈ClNO₂.HBr), A77636 ((1R,3S)-3-(1-adamantyl)-1-(aminomethyl)-3,4-dihydro-1H-isochromene-5,6-diol), A68930 ((1R,3S)-1-(aminomethyl)-3-phenyl-3,4-dihydro-1H-isochromene-5,6-diol), SKF 82526 (fenoldopam), and racemic trans-10,11-dihydroxy-5,6,6a,7,8,12b-hexahydrobenzo[a]phenanthridine.

For example, a dopamine D₂ agonist can be any one or more of those compounds known to those skilled in the art that are capable of activating or potentiating D₂ dopamine receptors. Dopamine D₂ agonists suitable for use in the present invention include, e.g., butyrophenones (e.g., spiroperidol), LY-171555 (quinpirole), bromocriptine, apomorphine, 2,10,11-trihydroxyapomorphine, lisuride, 2-OH-NPA ((R)-2,10,11-trihydroxy-N-propylnoraporphine), (−) -MDO-NPA (C₂₀H₂₁NO₂.xHCl), and propylnorpamorphine HCl R(−)-(NPA). A preferred class of D₂ agonists includes ergot alkaloids, e.g., 2-bromo-alpha-ergocriptine (bromocriptine), dihydroergocriptine, dihydroergotoxin, hydergene, dihydroergotamine, 6-methyl-8-beta-carbobenzyloxyaminoethyl-10-alpha-ergoline, 8-acylaminoergoline, 6-methyl-8-alpha-(N-acyl)amino-9-ergoline, lisuride, (6-methyl-8-alpha-N-phenyl-acetyl)amino-9-ergoline, ergocornine, 9,10-dihydroergocornine, any D-2-halo-6-alkyl-8-substituted ergoline, and D-2-bromo-6-methyl-8-cyanomethylergoline.

Effective amounts of ergot alkaloid for humans and vertebrates when administered alone (not conjoined to a D₁ agonist) are typically within the range of about 0.5 μg/kg body weight/day to about 0.2 mg/kg body weight/day. However, the amount of the D₁ agonist or the D₂ agonist depends on the condition being treated, the route of administration chosen, and the specific activity of the compound used and ultimately will be decided by the attending physician or veterinarian.

In general, effective amounts of dopamine D₂ agonist for humans and vertebrates are within the range of about 2 μg/kg body weight/day to about 3.5 mg/kg body weight/day.

Agents that increase the brain dopaminergic neuronal activity are optimally administered to a human or vertebrate so as to effectuate a peak in brain dopaminergic neuronal activity within about 4 hours of the onset of the human or vertebrate's daily waking cycle. For example, if the subject is a diurnal animal (e.g., a human), agents that increase the brain dopaminergic neuronal activity can optimally be administered to the subject so as to effectuate a peak in brain dopaminergic neuronal activity between the time interval of about 0400 and about 1200 hours of the day.

One or more of the agents disclosed herein can be administered, e.g., orally, parenterally, by inhalation spray or nebulizer, topically, rectally, nasally, buccally, vaginally, via an implanted reservoir, by injection (e.g., intravenously, intra-arterially, subdermally, intraperitoneally, intramuscularly, and/or subcutaneously), in an ophthalmic preparation, or via transmucosal administration. Suitable dosages may range from about 0.001 to about 50 mg/kg body weight, or according to the requirements of the particular drug. The agents can be administered in conjunction with any conventional non-toxic pharmaceutically-acceptable carriers, adjuvants or vehicles. The term parenteral as used herein includes subcutaneous, intracutaneous, intravenous, intramuscular, intraarticular, intra-arterial, intrasynovial, intrasternal, intrathecal, intralesional and intracranial injection or infusion techniques. Alternatively, or in addition, the present invention may be administered according to any of the Food and Drug Administration approved methods, for example, as described in the FDA Data Standards Manual (DSM) (available at http://www.fda.gov/Drugs/DevelopmentApprovalProcess/FormsSubmissionRequirements/ElectronicSubmissions/DataStandardsManualmonographs).

The term “pharmaceutically-acceptable carrier or adjuvant” refers to a carrier or adjuvant that may be administered to a subject, together with a compound of this invention, and which does not destroy the pharmacological activity thereof and is nontoxic when administered in doses sufficient to deliver a therapeutic amount of the compound.

Pharmaceutically-acceptable carriers, adjuvants and vehicles that can be used in the pharmaceutical compositions of this invention include, but are not limited to, e.g., ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of one or more compounds described herein.

The following Examples are intended to illustrate the inventions and details thereof, but they are not intended to limit the scope of the invention.

### Example 1

An investigation was conducted assessing the effect of BQR (1.6 to 4.8 mg/day) versus placebo given within two hours of waking in the morning for a period of 24 weeks on RHR and the relationship of this effect with its glycemic control effect in type 2 diabetes subjects from the Cycloset® Safety Trial (Gaziano J M et al, Diabetes Care 33: 1503-1508, 2010) with baseline HbA1c≥7.5% on 1-2 oral hypoglycemic agents with no concomitant diabetes or blood pressure medication change at 24 weeks. RHR was obtained using an echocardiogram (EKG or ECG). Multivariate regression and categorical analyses with cutoffs at baseline RHR< or ≥70 beats per minute were conducted. Among subjects with baseline RHR≥70 (N=61 BQR, 30 placebo; 2:1 randomization), multivariate regression revealed that baseline RHR is a significant positive predictor of BQR (but not placebo) induced RHR reduction (β=−0.30, P=0.02). BQR reduced RHR by 5.5 beats per minute versus placebo in subjects with baseline RHR≥70 (mean RHR change: −4 beats per minute BQR, 1.5 beats per minute placebo; p=0.003) and by 9 beats per minute in subjects with baseline RHR≥80 (N=32; mean RHR change: −7 beats per minute BQR, 2 beats per minute placebo; p=0.004) without change in blood pressure. With baseline RHR<70 (N=92), there was no significant change (either within or between groups) in RHR. Analysis of HbA1c reduction as a function of bRHR demonstrated HbA1c reductions with BQR versus placebo as follows: <70 beats per minute: −0.62 (P=0.009); ≥70 beats per minute: −0.71 (P=0.007); ≥80 beats per minute: −1.13 (P=0.01). Furthermore, in subjects with baseline RHR≥70, multivariate regression analyses demonstrated that the magnitude of RHR reduction is an independent predictor of the magnitude of HbA1c reduction with BQR therapy but not placebo (β=0.47, P=0.001). Therefore, dopamine agonist therapy that effectuates a circadian peak in brain dopaminergic neuronal activity with 4 hours of waking in humans (i.e., generally at the time of the circadian peak in central dopaminergic neuronal activity in healthy individuals) produces an improvement in elevated RHR and hyperglycemia in T2DM subjects. There are no anti-diabetes agents currently FDA approved that reduce both elevated heart rate and hyperglycemia in T2DM subjects.

### Example 2

An investigation was conducted to evaluate whether treatment with BQR reduces cardiovascular indices of elevated sympathetic tone, expressed as simultaneous reduction of blood pressure (BP) and elevated RHR. Sympathetic nervous system overactivity is a known risk factor for cardiovascular disease (CVD), a leading cause of mortality in type 2 diabetes. High resting heart rate (RHR, ≥70 beats per minute (BPM), a marker of elevated sympathetic tone, is considered a strong predictor of CVD outcomes. BQR, a sympatholytic, dopamine D₂ agonist, insulin sensitizer therapy for type 2 diabetes, has been observed to reduce CVD events. BQR's effect on RHR, systolic/diastolic BP (SBP, DBP) and mean arterial pressure (MAP) were analyzed in a large cohort of type 2 diabetes subjects (N=1502: 949 BQR, 553 P), randomized to BQR (1.6 to 4.8 mg/day) vs placebo (P) administered within 2 hours of waking in the morning and added to standard therapy (diet alone or ≤2 diabetes medications) from the Cycloset Safety Trial, referenced above, completing 24 weeks of study drug with no concomitant diabetes or hypertension medication changes. Baseline study group demographics were similar. Analyses were stratified by baseline RHR (bRHR)< or ≥70 BPM. As a secondary analysis, BQR's glycemic effect was assessed as the percentage of subjects with baseline HbA1c (A1c)>7 (N=505: 321 BQR, 184 P) achieving A1c≤7 at 24 weeks. In subjects with bRHR≥70 (N=582: 372 BQR; 210 P), BQR (vs P) reduced RHR by −2.5 BPM (−4.5 BQR, −1.97 P, p=0.001), SBP by −4.1 mmHg (−3.5 BQR, 0.6 P, p=0.001), DBP by −2.4 mmHg (−2.8 BQR, −0.4 P, p=0.004) and MAP by −2.95 mmHg (−3.0 BQR, −0.07 P, p=0.001). Subjects with bRHR<70 had no significant change in RHR or BP. The percentage of subjects achieving A1c<7 with BQR was higher by 2.2 fold (BQR 41%, P 19%; p<0.001) and 3.7 fold (37% BQR, 10% P, p<0.001) in the bRHR< and ≥70 groups respectively. Therefore, in addition to improving glycemic control, BQR lowers hemodynamic parameters of elevated sympathetic tone.

Based on preclinical and clinical evidence, it is postulated that BQR administration within two hours of waking, restores the normal circadian peak in central dopaminergic activity that is diminished in type 2 diabetes.

### Example 3

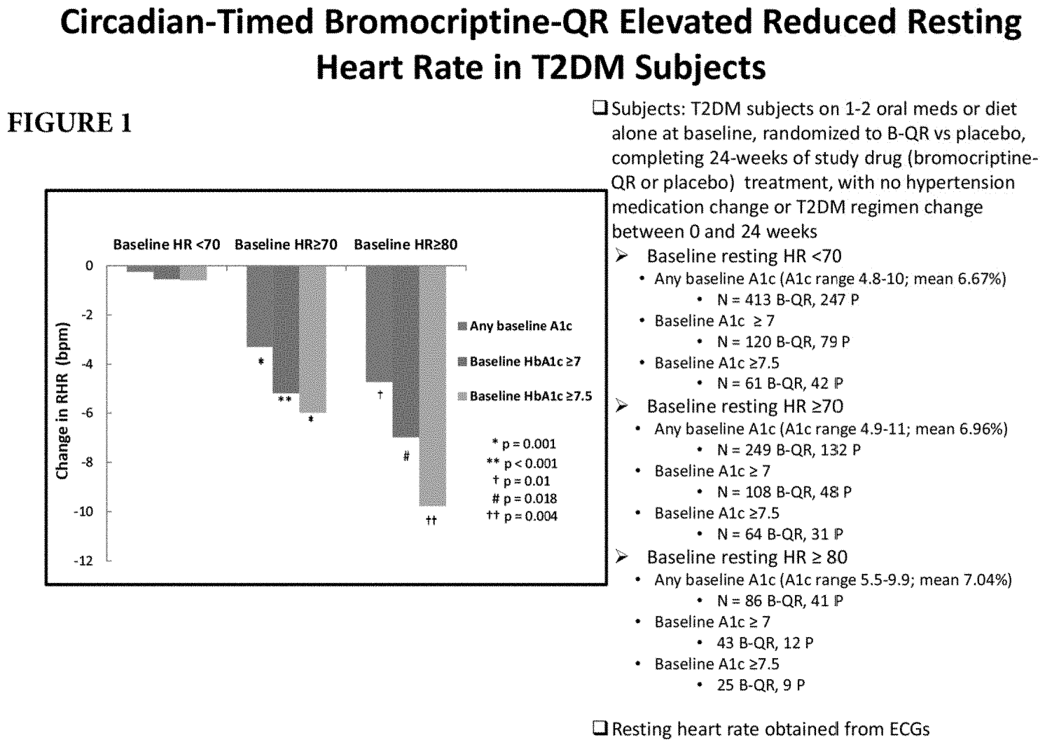

An investigation was conducted to evaluate whether treatment with BQR reduces elevated RHR as a function of baseline RHR and as a function of baseline glycemic control in T2DM subjects. BQR, a sympatholytic, dopamine D₂ agonist, insulin sensitizer therapy for type 2 diabetes, has been observed to reduce CVD events. BQR's effect on RHR were analyzed in a large cohort of type 2 diabetes subjects (N=1502: 949 BQR, 553 P), randomized to BQR (1.6 to 4.8 mg/day) vs placebo (P) administered within 2 hours of waking in the morning and added to standard therapy (diet alone or ≤2 diabetes medications) from the Cycloset Safety Trial, referenced above, completing 24 weeks of study drug with no concomitant diabetes or hypertension medication changes. Baseline study group demographics were similar. Analyses were stratified by baseline RHR (bRHR)<70 BPM, ≥70 BPM or >/=80 BPM and by HBa1c (any incoming HbA1c, HbA1c>/=7.0, and HbA1c>/=7.5). The results displayed in FIG. 1 demonstrate that such BQR therapy significantly reduces elevated RHR but not normal RHR and that such reduction is positively correlated to the baseline RHR and inversely correlated with the degree of glycemic control at baseline (e.g., baseline HbA1c level) in these study subjects (i.e., the effect to reduce elevated RHR increases as elevated RHR increases and also as baseline HbA1c increases (increasing deterioration of glycemic control)).

Dopamine agonist therapy has been documented to both increase and decrease heart rate in several experimental paradigms of animal and human studies. This inconsistency precludes its use in any therapy related to long term heart rate modification. It has been found that to achieve a consistent and potent reduction in elevated resting heart rate, particularly in subjects with metabolic disease, and more particularly in subjects with poor glycemic control (HbA1c>1=7.0) with dopamine activity modulating therapy, the therapy must be applied in a manner that reestablishes the central circadian dopaminergic neuronal activity back towards that observed in normal healthy individuals of the same species and sex. When this approach is employed the circadian dopamine agonist therapy does not alter RHR if it is in the normal range, but does consistently reduce elevated RHR. Surprisingly, it has been found that elevated resting heart rate serves as a biomarker of individuals that will have an enhanced responsiveness to such dopamine neuronal activity modulation in the effective treatment of metabolic disease. In other words, as the degree of elevated heart rate increases among individuals with metabolic disease so does the beneficial metabolic responsiveness to circadian dopamine neuronal activity modulating therapy (i.e., reestablishing the normal circadian peak in central dopaminergic neuronal activity) in treating the metabolic disease (and the elevated heart rate). This correlation does not exist among subjects without elevated heart rate. Consequently, heart rate effects on the overall population (all RHR measures from all subjects) cannot be utilized as a measure or indicator of this correlation or as an identification method of responders to circadian dopamine neuronal activity modulation therapy for metabolic disease. Elevated RHR among subjects with metabolic disease identifies subjects most likely to receive the most benefit from such circadian dopamine neuronal activity modulation (i.e., reestablishing the normal circadian peak in central dopaminergic neuronal activity). Moreover, it has been surprisingly found that the severity of metabolic disease, e.g., level of hyperglycemia (e.g., elevated HbA1c level) in type 2 diabetes, identifies those individuals likely to best respond to the effect of such dopamine activity modulating therapy for reducing elevated resting heart rate.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-18. (canceled)

19. A method of improving HbA1c responsiveness to treatment with bromocriptine in a subject suffering from type-2 diabetes and having an elevated HbA1c level, the method comprising:
a. identifying a subject suffering from type-2 diabetes;
b. determining that the subject suffering from type-2 diabetes has an elevated HbA1c level of 7.5% or greater;
c. determining that the resting heart rate of the subject suffering from type-2 diabetes and having an elevated HbA1c level is greater than or equal to 70 beats per minute; and
d. administering to the subject bromocriptine at a dosage of between 1.6 and 4.8 mg/day, wherein the subject experiences a greater decrease in HbA1c levels compared to a subject suffering from type-2 diabetes and presenting with the same elevated HbA1c level and a resting heart rate of less than 70 beats per minute.

20. The method of claim 19, wherein the subject's resting heart rate is greater than or equal to 80 beats per minute.

21. A method of improving HbA1c responsiveness to treatment with bromocriptine in a subject suffering from type-2 diabetes, the method comprising:
a. identifying a subject suffering from type-2 diabetes;
b. determining that the subject suffering from type-2 diabetes has an elevated HbA1c level of 7.5% or greater;
c. determining that the resting heart rate of the subject suffering from type-2 diabetes and having an elevated HbA1c level is greater than or equal to 70 beats per minute;
d. administering to the subject bromocriptine at a dosage of between 1.6 and 4.8 mg/day; and
e. determining the subject's resting heart rate after administration of the bromocriptine, wherein the subject experiences a greater decrease in HbA1c levels compared to a subject suffering from type-2 diabetes and presenting with the same elevated HbA1c level and a resting heart rate of less than 70 beats per minute.

22. The method of claim 19, wherein the bromocriptine is administered to the subject so as to effectuate a peak in brain dopaminergic neuronal activity within 4 hours of the onset of the subject's daily waking cycle.

23. The method of claim 19, wherein the subject is a diurnal animal and the bromocriptine is administered to the subject so as to effectuate a peak in brain dopaminergic neuronal activity between the time interval of 0400 and 1200 hours of the day.

24. A method of method of improving HbA1c responsiveness to treatment with bromocriptine in a subject suffering from type-2 diabetes and having an elevated HbA1c level, the method comprising:
a. identifying a subject suffering from type-2 diabetes;
b. determining that the subject suffering from type-2 diabetes has an elevated HbA1c level of 7.5% or greater;
c. determining that the resting heart rate of the subject suffering from type-2 diabetes and having an elevated HbA1c level is greater than or equal to 70 beats per minute; and
d. administering to the subject bromocriptine at a dosage of between 1.6 and 4.8 mg/day, wherein the subject experiences a greater decrease in HbA1c levels compared to a subject suffering from type-2 diabetes and presenting with the same elevated HbA1c level and a resting heart rate of less than 70 beats per minute.
